(19) United States
(12) Patent Application Publication
(10) Pub. No.: US 2014/0005689 A1
(43) Pub. Date: Jan. 2, 2014

(54) SUTURE CUTTER

(71) Applicant: JERRY R. GRIFFITHS, NORWELL, MA (US)

(72) Inventor: JERRY R. GRIFFITHS, NORWELL, MA (US)

(73) Assignee: SYMMETRY MEDICAL NEW BEDFORD, INC, NEW BEDFORD, MA (US)

(21) Appl. No.: 13/534,378

(22) Filed: Jun. 27, 2012

(51) Int. Cl. A61B 17/06

(52) U.S. Cl. 606/138

(57) ABSTRACT

An improved suture cutter for cutting high strength sutures used in arthroscopic surgeries includes a movable handle that moves rotationally around a pivot. This rotational motion is translated through a linkage into near linear movement at the distal end of a moving member. At the distal end, the moving member pushes a cutting blade onto an inclined stationary blade and thereby cuts a suture captured between the moving cutting blade and the inclined stationary blade.

### FIELD OF THE INVENTION

The present invention relates to a suture cutter, and more particularly to a suture cutter for cutting high strength sutures.

### BACKGROUND OF THE INVENTION

Suture cutters usually include one cutting blade pressed onto an anvil or a cradle, or two cutting blades sliding past each other and being held together with mechanical means. Most of these suture cutters are suitable for cutting standard sutures such as braided silk or polypropylene sutures. In some cases, however, ultra high strength sutures made of abrasive or otherwise tough materials are used for fastening tissue to bone or to other tissue. Cutting ultra high strength sutures with conventional suture cutters is not efficient and frequently results in unacceptable fraying of the suture ends. Accordingly, there is a need for an improved suture cutting mechanism suitable for cutting high strength sutures, such as sutures made of ultra-high molecular weight polyethylene (UHMWPE).

### SUMMARY OF THE INVENTION

The present invention describes an improved mechanism for cutting high strength sutures used in arthroscopic surgeries. The suture cutting device includes a movable handle that moves rotationally around a pivot. This rotational motion is translated through a linkage into near linear movement at the distal end of a moving member. At the distal end, the moving member pushes a cutting blade onto an inclined stationary blade and thereby cuts a suture captured between the moving cutting blade and the inclined stationary blade.

In general, in one aspect, the invention features a suture cutting instrument including a handle assembly, a driver, and a suture cutting assembly. The handle assembly includes a stationary handle and a movable handle pivotally connected to the stationary handle at a pivot and the movable handle moves rotationally around the pivot. The driver has a proximal end connected to the movable handle and is configured to reciprocate longitudinally when the movable handle moves rotationally around the pivot. The suture cutting assembly includes a movable cutting blade and an inclined stationary blade. The movable cutting blade is connected to a distal end of the driver and is configured to move longitudinally onto the inclined stationary blade when the movable handle moves rotationally around the pivot and thereby to cut a suture captured between the movable cutting blade and the inclined stationary blade.

Implementations of this aspect of the invention may include one or more of the following features. The suture cutting assembly further includes a suture capture component and the suture capture component includes a hook. The suture cutting instrument further includes an elongated cannula and the driver is configured to move longitudinally within the cannula. The suture capture component is attached to the distal end of the elongated cannula. The suture capture component is removably attached to the distal end of the elongated cannula. The suture capture component has a cylindrical body having an axial through opening and being coaxial with the elongated cannula. The cylindrical body has cylindrical top and bottom outer surfaces and flat first and second side surfaces. The bottom surface includes a first oval-shaped opening and the top surface includes an elongated slot. The front end of the cylindrical body includes a second oval-shaped opening, the hook and the inclined stationary blade. The second oval-shaped opening and the hook are set apart at a distance dimensioned to allow passage and capture of the suture by the hook. The movable cutting blade comprises a front end having an angled blade surface and the angled blade surface is configured to slide up and down the inclined stationary blade. The handle assembly further includes a lever mechanism configured to secure the movable handle at a predetermined distance relative to the stationary handle. The lever mechanism includes a lever terminating at one end at a lever button and having an opposite end pivotally connected to the stationary handle via a pivot pin and a spring mechanism. The lever further includes a notch configured to engage a matching opposite notch in the back side of the movable handle and thereby to prevent movement of the movable handle.

In general, in another aspect, the invention features a method for cutting a suture including the following. First, providing a handle assembly comprising a stationary handle and a movable handle pivotally connected to the stationary handle at a pivot. The movable handle moves rotationally around the pivot. Next, providing a driver comprising a proximal end connected to the movable handle and being configured to reciprocate longitudinally when the movable handle moves rotationally around the pivot. Next, providing a suture cutting assembly comprising a movable cutting blade and an inclined stationary blade. The movable cutting blade is connected to a distal end of the driver and is configured to move longitudinally onto the inclined stationary blade when the movable handle moves rotationally around the pivot and thereby to cut a suture captured between the movable cutting blade and the inclined stationary blade.

Among the advantages of this invention may be one or more of the following. When the moving blade meets resistance in cutting the high strength suture, the force applied at the handle results in the moving blade being forced against the inclined stationary blade and this results in improving the shearing of the suture. The suture cutting mechanism may be applied for cutting high strength sutures, such as sutures made of ultra-high molecular weight polyethylene (UHMWPE). The suture cutting mechanism may also be used for cutting sutures made of biocompatible materials, Nylon, Dacron, Polypropylene, silk, or braided sutures, among others. The suture cutting mechanism may also be applied in micro-scissors and other applications where two surfaces need to be kept from separating.

The details of one or more embodiments of the invention are set forth in the accompanying drawings and description below. Other features, objects and advantages of the invention will be apparent from the following description of the preferred embodiments, the drawings and from the claims.

### BRIEF DESCRIPTION OF THE DRAWINGS

Referring to the figures, wherein like numerals represent like parts throughout the several views:

FIG. 1 is a perspective view of an endoscopic instrument with a suture cutting front end assembly;

FIG. 2A is a partial exploded view of the endoscopic instrument of FIG. 1;

FIG. 2B is detailed view of the actuator rod connection to the movable handle of FIG. 2A;

FIG. 3A is a partial exploded view of the suture cutting front end assembly of FIG. 1;

FIG. 3B is a cross-sectional view of the suture cutting front end assembly along AA plane, shown in FIG. 4A;

FIG. 4A is a top view of the endoscopic instrument of FIG. 1;

FIG. 4B is a side view of the endoscopic instrument of FIG. 1;

FIG. 4C is a detailed side view of the handle of the endoscopic instrument of FIG. 1;

FIG. 5A is a side view of the endoscopic instrument of FIG. 1 with the front end assembly in the “open” position;

FIG. 5B is a detailed view of the suture cutting front end assembly of FIG. 5A;

FIG. 6A is a side view of the endoscopic instrument of FIG. 1 with the front end assembly in the “suture captured” position;

FIG. 6B is a detailed view of the suture cutting front end assembly of FIG. 6A;

FIG. 7A is a side view of the endoscopic instrument of FIG. 1 with the front end assembly in the “closed” position;

FIG. 7B is a detailed view of the suture cutting front end assembly of FIG. 7A;

FIG. 8A is a detailed view of the suture cutting front end assembly of FIG. 1 in the “open” position;

FIG. 8B is a detailed view of the suture cutting front end assembly of FIG. 1 in the “suture captured” position;

FIG. 8C is a detailed view of the suture cutting front end assembly of FIG. 1 in the “closed” position;

FIG. 9A is a detailed cross-sectional view of the suture cutting front end assembly of FIG. 1 in the “open” position;

FIG. 9B is a detailed cross-sectional view of the suture cutting front end assembly of FIG. 1 in the “suture captured” position;

FIG. 9C is a detailed cross-sectional view of the suture cutting front end assembly of FIG. 1 in the “closed” position;

FIG. 10A is a detailed top view of the suture cutting front end assembly of FIG. 1 in the “closed” position;

FIG. 10B is a detailed side view of the suture cutting front end assembly of FIG. 1 in the “closed” position; and

FIG. 10C is a detailed bottom view of the suture cutting front end assembly of FIG. 1 in the “closed” position;

### DETAILED DESCRIPTION OF THE INVENTION

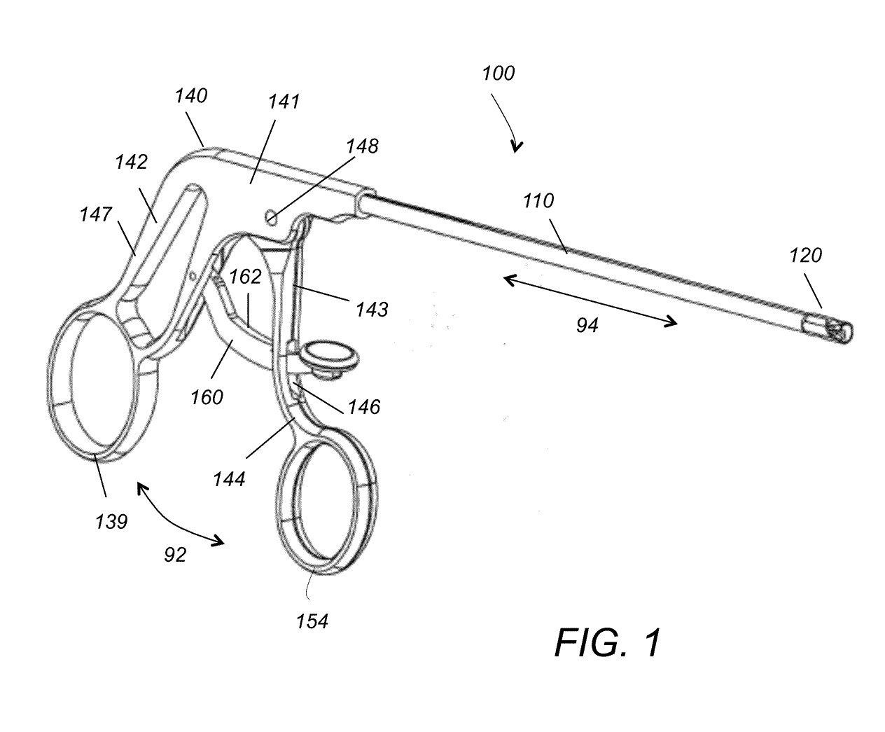

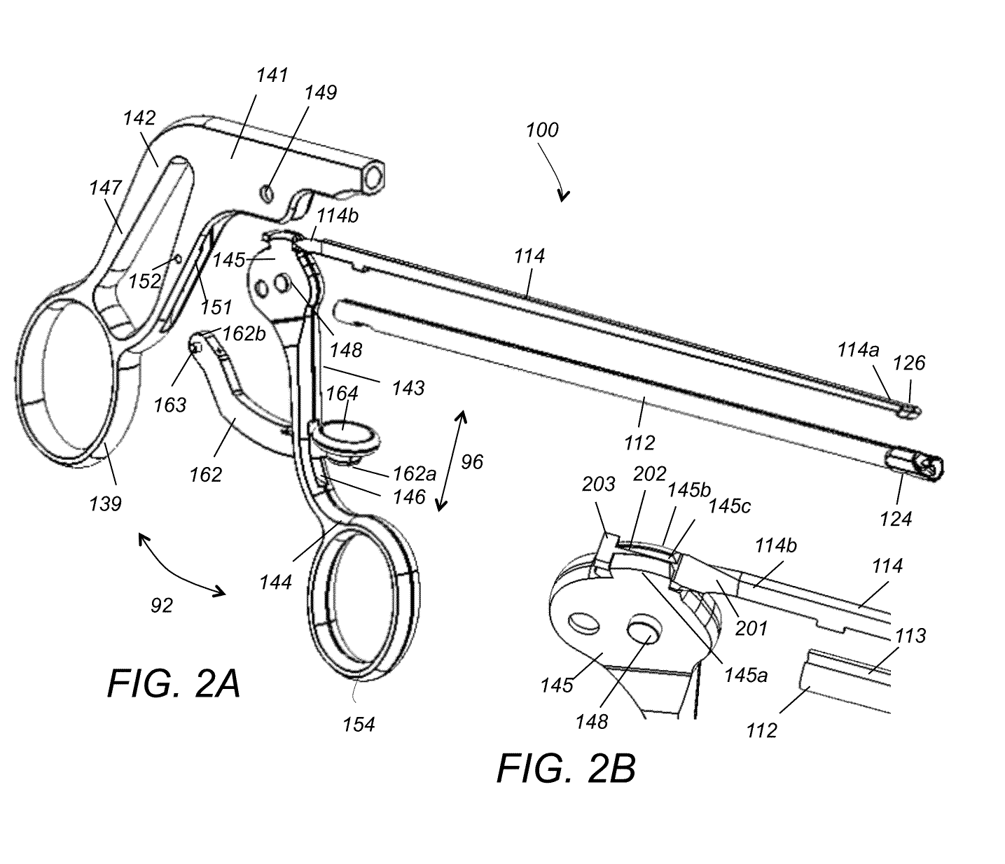

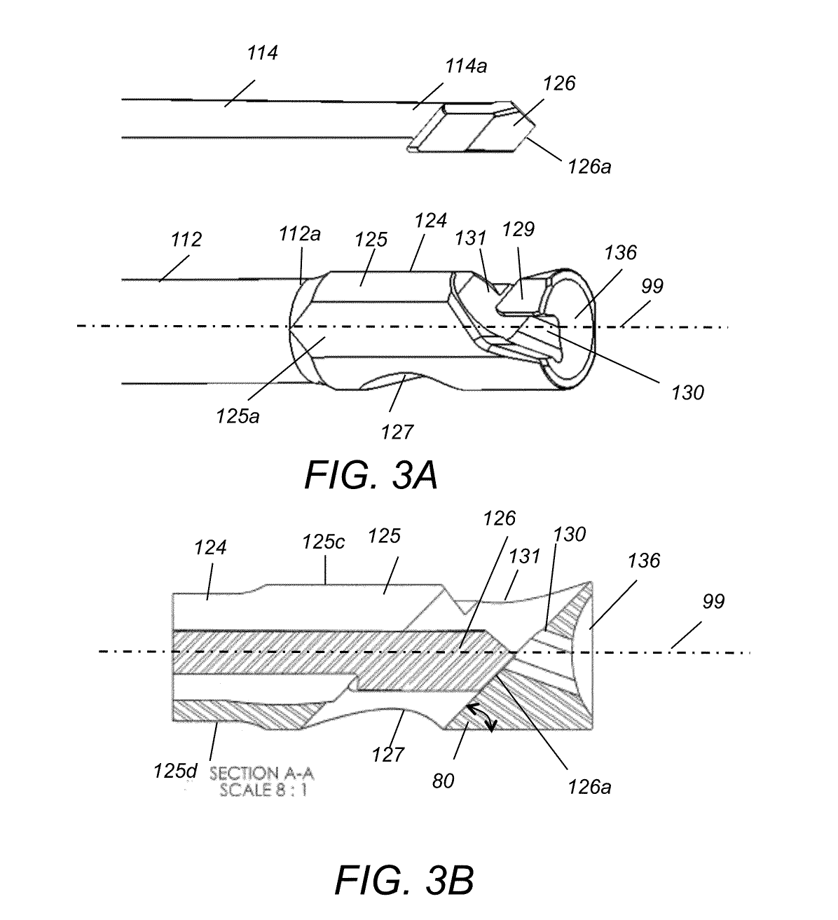

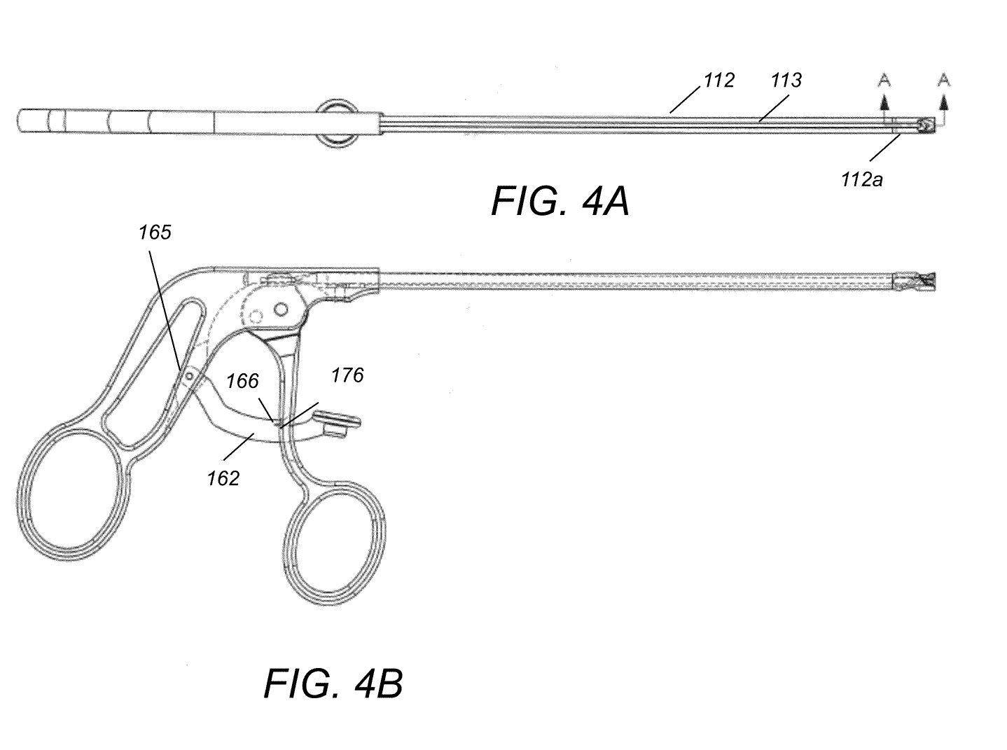

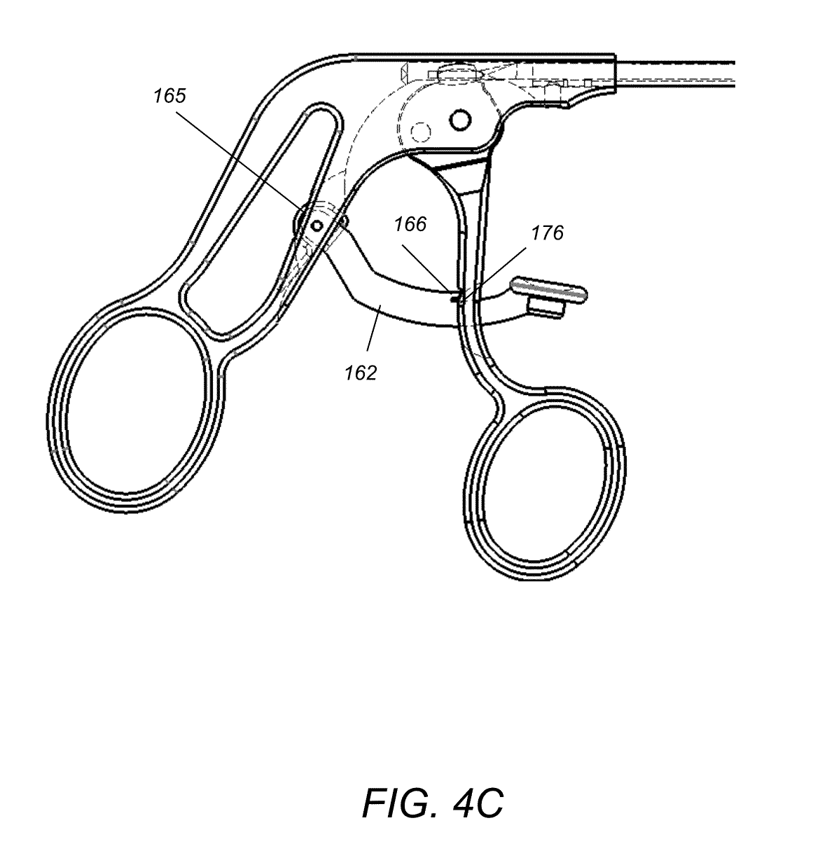

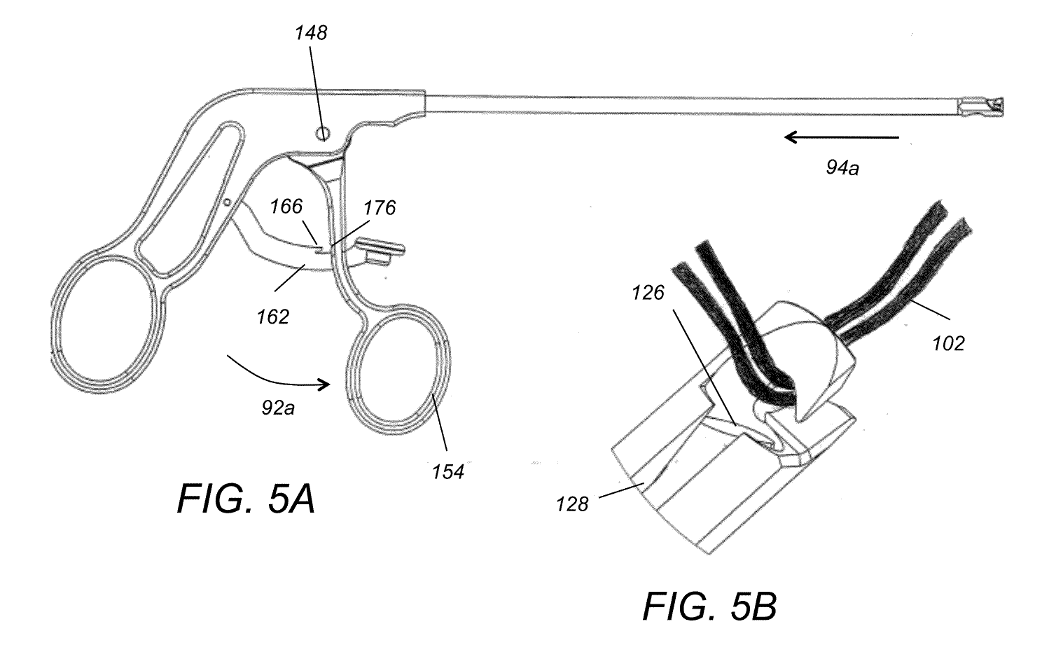

Referring to FIG. 1, FIG. 2, FIG. 3A and FIG. 3B, the endoscopic instrument 100 includes a suture cutting front end assembly 120, a cannula/actuator rod assembly 110, and a handle assembly 140. The suture cutting assembly 120 includes a suture capture component 124 and a blade 126. The cannula/actuator rod assembly 110 includes an outer cannula 112 and an actuator rod 114 configured to move linearly within the cannula 112. Cannula 112 has an elongated slot 113 extending the entire length of the cannula 112 and a front end 112a forming the suture capture component 124 of the suture cutting assembly 120. In this embodiment, the suture capture component 124 is an integral part of the cannula front end 112a. In other embodiments, the suture capture component 124 is removably attached to the front end of the cannula. Actuator rod 114 had a distal end 114a attached to blade 126, as shown in FIG. 3A, and a proximal end 114b attached to the movable handle 144, as shown in FIG. 2B and will be described below. The handle assembly 140 includes a stationary handle 142, a movable handle 144, and a base 141. In this embodiment, the stationary handle 142 is integral with and extends downward from the base 141 and movable handle 144 is pivotally attached to the base 141 via pivot pin 148. Movable handle 144 is secured at a predetermined distance relative to the stationary handle 142 via a lever mechanism 160. Movable handle 144 is also connected to the blade 126 via the actuator rod 114. Actuator rod 114 translates the rotational motion 92 of the movable handle 144 into a linear motion 94 of the blade 126. Movable handle 144 includes a finger loop 154, an elongated curved middle segment 143, and an upper segment 145. Middle segment 143 includes a notch 176 and a slot 146, which is shaped and dimensioned to accommodate lever 162, as shown in FIG. 2A, FIG. 4B, FIG. 4C and FIG. 5A. Upper segment 145 includes two upward extending protrusions 145a, 145b that are parallel to each other and form a slot 145c between them. The slot 145c is dimensioned to receive end 114b of the actuator rod. End 114b includes a vertically oriented elongated tetrahedron 201, a first parallelepiped block 202 extending horizontally from the front surface of the tetrahedron 201 and a second parallelepiped block 203 extending from a front surface of the first block 202 vertically and coplanar with the first block 202. The first block 202 is dimensioned to fit within slot 145c. Forward or backward motion of the actuator rod 114 relative to the upper segment 145 of the movable handle 144 is prevented by second block 203 and tetrahedron 201, respectively. Lever mechanism 160 includes a curved lever 162 terminating in one end 162a at a lever button 164 and having an opposite second end 162b pivotally connected to stationary handle 142 via a pivot pin 163 and a spring mechanism 165, as shown in FIG. 2A, FIG. 4B and FIG. 4C. Spring mechanism 165 causes lever end 162a to move up along direction 96 when lever button 164 is not pressed down. Lever end 162a also includes a notch 166 that engages a matching opposite notch 176 in the back side of movable handle 144 and acts as a stop of the movable handle 144 motion 92 when the lever button 164 is not pressed down. In this position the front end assembly is in the “open” position, as will be described below. Notch 166 is located at a distance from lever end 162a thereby allowing the movable handle 144 to rotate slightly back toward the stationary handle 142. Moving the movable handle 144 slightly back toward the stationary handle 142 causes the actuator rod 114 and the attached blade 126 to move slightly forward and to bring the front end assembly 120 in the “suture capture” position. Pressing the lever button 164 down, disengages the movable handle 144 from the lever notch 166 and allows it to rotate further back toward the stationary handle 142. Moving the movable handle 144 further back toward the stationary handle 142 causes the actuator rod 114 and the attached blade 126 to move further forward and to bring the front end assembly in the “closed” position. Stationary handle 142 includes a finger loop 139 a middle segment 147 and base 141 that includes a through opening 149 dimensioned to receive a pivot pin 148 for pivotally attaching the movable handle 144 to the stationary handle 142. Middle segment 147 includes a slot 151 that is configured and dimensioned to accommodate the second end 162b of lever 162. Second end 162b of lever 162 includes a pivot pin 163 that is configured to be received within through opening 152 formed in the middle segment 147 of stationary handle 142.

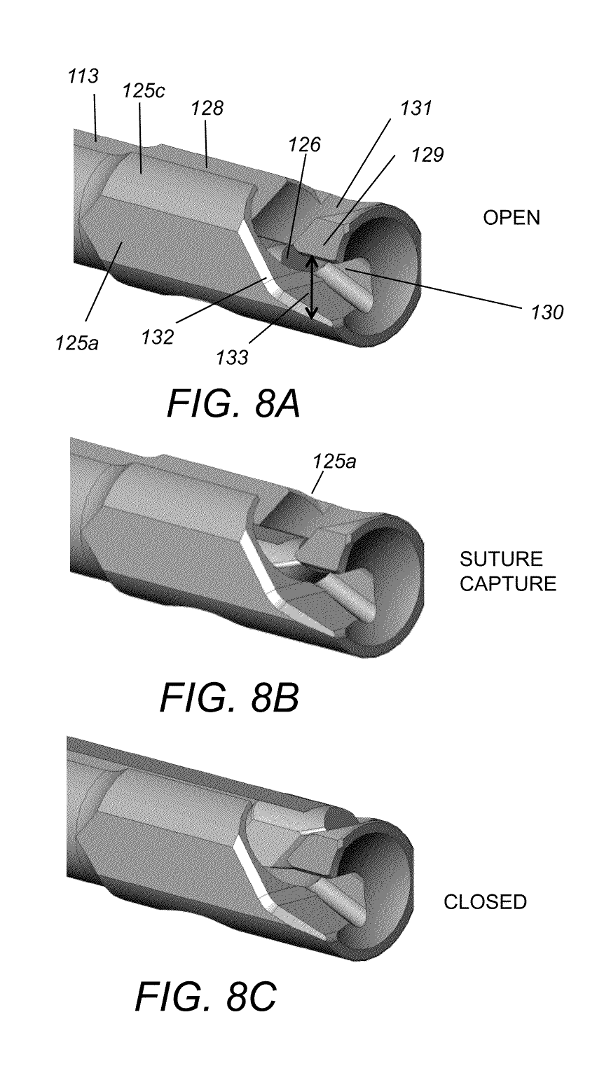

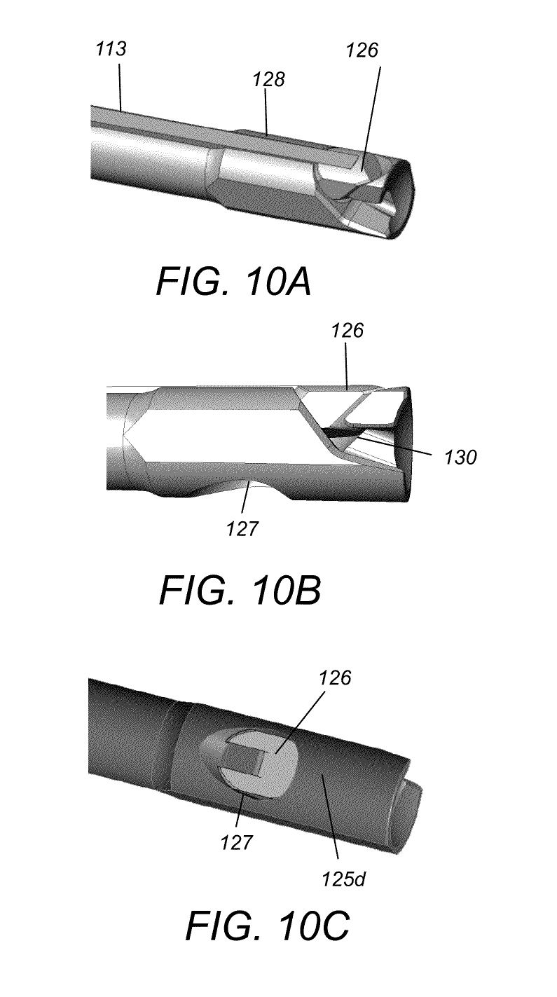

Referring to FIG. 3A and FIG. 3B, the suture capture component 124 includes a cylindrical body 125 attached to the distal end 112a of the outer cannula 112. In this embodiment, cylindrical body 125 is integral with the distal end 112a of the cannula 112 and includes an axial through opening 136 extending along cannula axis 99. Cylindrical body 125 includes two opposite flat side surfaces 125a, 125b (shown in FIG. 8B) and cylindrical top and bottom surfaces 125c, 125d, respectively. Bottom 125d surface includes an oval shaped opening 127 and top surface 125c includes an elongated slot opening 128 which is a continuation and an extension of the outer cannula elongated slot 113, as shown in FIG. 10A. The front end of cylindrical body 125 includes an oval-shaped opening 131 with an integrated hook 129 and an inclined stationary blade surface 130 forming an angle 80 relative to the cannula axis 99. Oval opening 131 extends down on side 125a of the cylindrical body from the top surface 125c toward the bottom surface 125d and forms a curved surface 132, as shown in FIG. 8A. Curved surface 132 and hook 129 are set apart at a distance 133, which is dimensioned to allow the passage and capture of the suture 102 by the hook 129. Actuator rod 114 with the attached blade 126 is dimensioned to move linearly within opening 136 as a result of the rotational pivoting motion of the movable handle 144. The front end of blade 126 includes an angled blade surface 126a which is configured to slide up and down the stationary inclined blade surface 130 as the actuator moves forward along direction 94 and backward, respectively. This sliding motion of the blade surface 126a onto inclined stationary blade surface 130 cuts the captured suture 102.

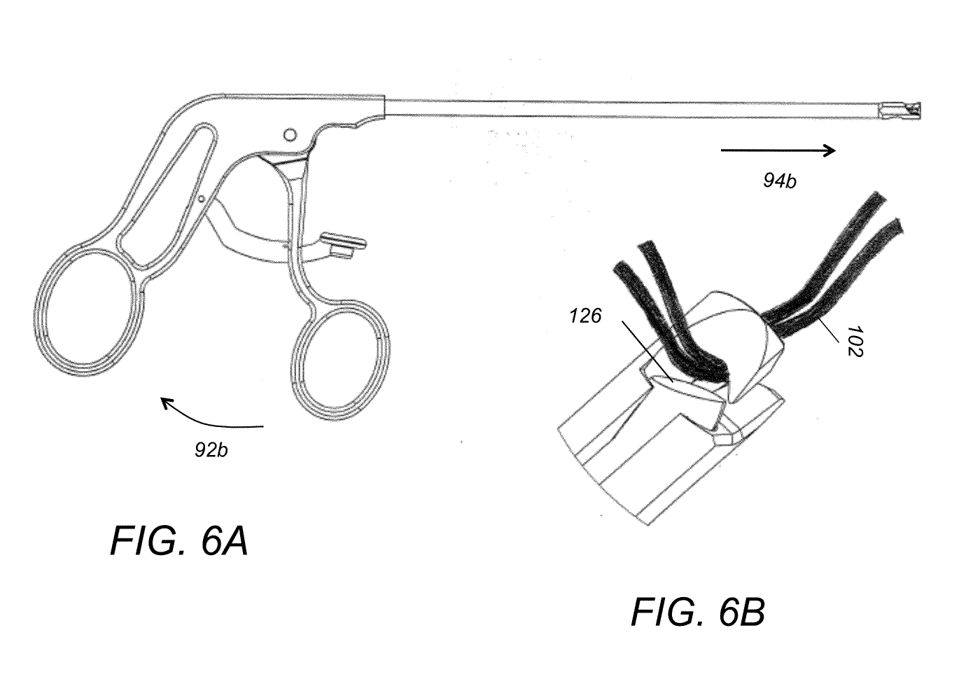

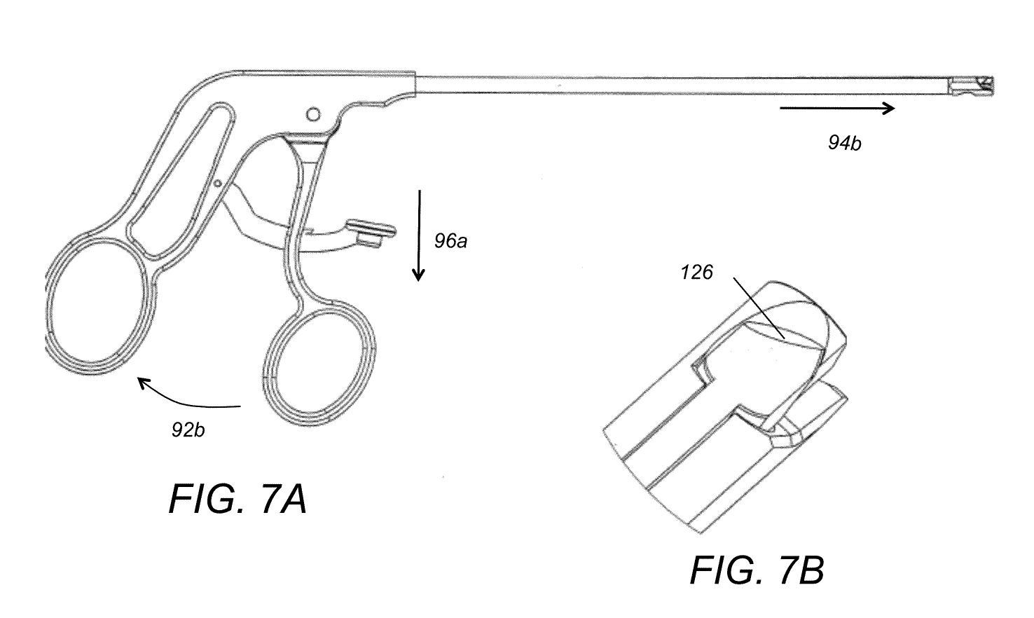

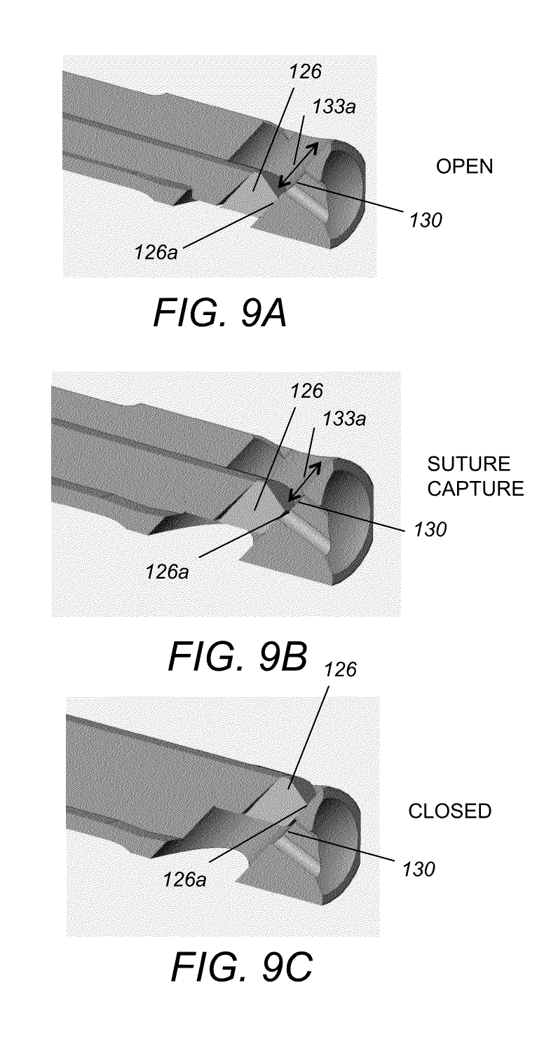

Referring to FIG. 5A, an operator moves the movable handle 144 away from the stationary handle 142, by placing his finger inside the finger loop 154 and pivoting the handle 144 around the pivot point 148 counter-clockwise. This pivoting motion 92a of the handle 144 away from the stationary handle 142 moves the actuator rod 114 back along direction 94a and brings blade surface 126a down at the bottom of the inclined stationary blade surface 130, as shown in FIG. 9A. This setting defines the “open” position, mentioned above and shown in FIG. 9A, FIG. 5A, FIG. 5B and FIG. 8A. At this point, the opening distance 133a between the top of the blade 126 and the top of the inclined stationary blade 130 is the largest and this permits the passage and engagement of the suture 102 by the hook 129, as shown in FIG. 5B. Next, the operator pivots the movable handle 154 slightly back clockwise along direction 92b until lever notch 166 engages handle notch 176 and stops the further pivoting of the movable handle 144, as shown in FIG. 6A. This slight back pivoting motion along 92b moves actuator rod 114 slightly forward 94b and causes blade surface 126a to move slightly up in the middle section of the inclined stationary blade surface 130, as shown in FIG. 9B. This setting defines the “suture capture” position, mentioned above and shown in FIG. 9B, FIG. 6A, FIG. 6B and FIG. 8B. At this point, the opening distance 133a between the top of blade 126 and the top of the inclined stationary blade 130 is reduced and this causes the capture of the suture 102 by the hook 129, as shown in FIG. 6B. Next, the operator presses lever button 164 down along direction 96a to disengage level notch 166 from the movable handle notch 176 and then pivots the movable handle 144 further back clockwise along direction 92b, as shown in FIG. 7A. This further back pivoting motion along 92b moves actuator rod 114 further forward 94b and causes blade surface 126a to move further up to the top section of the inclined stationary blade surface 130, as shown in FIG. 9C. This setting defines the “closed” position, mentioned above and shown in FIG. 9C, FIG. 7A, FIG. 7B and FIG. 8C. At this point, the opening distance 133a between the top of blade 126 and the top of the inclined stationary blade 130 is further reduced and this causes the cutting of the suture 102 by the blade 126, as shown in FIG. 7B. When cutting high strength sutures the moving blade surface 126a pushes away from the inclined stationary blade surface 130. If the gap between the top of the moving blade 126a and the top of the inclined stationary blade surface 130 is too large, it will result in an unacceptable fraying and/or a tag end at the ends of the cut suture. If the gap between the top of the moving blade 126a and the top of the inclined stationary blade surface 130 is even larger the suture will not be cut completely through. When the moving blade 126 meets resistance in cutting suture, the force applied at the handle 144 results in the moving blade 126 being forced against the stationary blade 130 and this improves the shearing of the suture.

In one example, the total stroke (i.e., the distance 133a between the blade top 126a and the top of the inclined blade surface 130) is about 0.123 inch and the clearance between the two blades is 0.00040 inch. The inclined stationary blade 130 may form an angle 80 with the cannula axis 99 in the range of 30 to 50 degrees. In the example of FIG. 3B, angle 80 is 45 degrees. In one example, the blade 126 has a diameter of 0.1248 inch, and the hook 129 dimensions are 0.030×0.054 inches. Typical dimensions for the handles are 0.230×4.5×1.75 inches. The suture cutting instrument may be made of various types of biocompatible stainless steels, ceramics, plastics, or composites. The suture may be made of ultra-high molecular weight polyethylene (UHMWPE), biocompatible material, Nylon, Dacron, Polypropylene, or braided sutures, among others. The end assembly may be disposable or non-disposable and may be made of various types of biocompatible stainless steels, metals, alloys, composites and plastics. The above described suture cutting mechanism may be applied in micro-scissors and other applications where two surfaces need to be kept from separating.

Several embodiments of the present invention have been described. Nevertheless, it will be understood that various modifications may be made without departing from the spirit and scope of the invention. Accordingly, other embodiments are within the scope of the following claims.

What is claimed is:

1. A suture cutting instrument comprising:
a handle assembly comprising a stationary handle and a movable handle pivotally connected to said stationary handle at a pivot and wherein the movable handle moves rotationally around the pivot;
a driver comprising a proximal end connected to the movable handle and being configured to reciprocate longitudinally when the movable handle moves rotationally around the pivot;
a suture cutting assembly comprising a movable cutting blade and an inclined stationary blade, wherein the movable cutting blade is connected to a distal end of the driver and is configured to move longitudinally onto the inclined stationary blade when the movable handle moves rotationally around the pivot and thereby to cut a suture captured between the movable cutting blade and the inclined stationary blade.

2. The suture cutting instrument of claim 1, wherein the suture cutting assembly further comprises a suture capture component.

3. The suture cutting instrument of claim 2, wherein said suture capture component comprises a hook.

4. The suture cutting instrument of claim 3, further comprising an elongated cannula and wherein said driver is configured to move longitudinally within said cannula.

5. The suture cutting instrument of claim 4, wherein said suture capture component is attached to the distal end of the elongated cannula.

6. The suture cutting instrument of claim 4, wherein said suture capture component is removably attached to the distal end of the elongated cannula.

7. The suture cutting instrument of claim 5, wherein said suture capture component comprises a cylindrical body comprising an axial through opening and being coaxial with the elongated cannula.

8. The suture cutting instrument of claim 7, wherein said cylindrical body comprises cylindrical top and bottom outer surfaces and flat first and second side surfaces and wherein the bottom surface comprises a first oval-shaped opening and the top surface comprises an elongated slot and wherein the front end of the cylindrical body comprises a second oval-shaped opening, said hook and said inclined stationary blade.

9. The suture cutting instrument of claim 8, wherein said second oval-shaped opening and said hook are set apart at a distance dimensioned to allow passage and capture of the suture by the hook.

10. The suture cutting instrument of claim 1, wherein said movable cutting blade comprises a front end having an angled blade surface and wherein said angled blade surface is configured to slide up and down the inclined stationary blade.

11. The suture cutting instrument of claim 1, wherein the handle assembly further comprises a lever mechanism configured to secure the movable handle at a predetermined distance relative to the stationary handle, and wherein the lever mechanism comprises a lever terminating at one end at a lever button and having an opposite end pivotally connected to the stationary handle via a pivot pin and a spring mechanism.

12. The suture cutting instrument of claim 11, wherein the lever further comprises a notch configured to engage a matching opposite notch in the back side of the movable handle and thereby to prevent movement of the movable handle.

13. A method for cutting a suture comprising:
providing a handle assembly comprising a stationary handle and a movable handle pivotally connected to said stationary handle at a pivot and wherein the movable handle moves rotationally around the pivot;
providing a driver comprising a proximal end connected to the movable handle and being configured to reciprocate longitudinally when the movable handle moves rotationally around the pivot;
providing a suture cutting assembly comprising a movable cutting blade and an inclined stationary blade, wherein the movable cutting blade is connected to a distal end of the driver and is configured to move longitudinally onto the inclined stationary blade when the movable handle moves rotationally around the pivot and thereby to cut a suture captured between the movable cutting blade and the inclined stationary blade.

14. The method of claim 13, wherein the suture cutting assembly further comprises a suture capture component.

15. The method of claim 14, wherein said suture capture component comprises a hook.

16. The method of claim 15, further comprising providing an elongated cannula and wherein said driver is configured to move longitudinally within said cannula.

17. The method of claim 16, wherein said suture capture component is attached to the distal end of the elongated cannula.

18. The method of claim 16, wherein said suture capture component is removably attached to the distal end of the elongated cannula.

19. The method of claim 17, wherein said suture capture component comprises a cylindrical body comprising an axial through opening and being coaxial with the elongated cannula.

20. The method of claim 19, wherein said cylindrical body comprises cylindrical top and bottom outer surfaces and flat first and second side surfaces and wherein the bottom surface comprises a first oval-shaped opening and the top surface comprises an elongated slot and wherein the front end of the cylindrical body comprises a second oval-shaped opening, said hook and said inclined stationary blade.

21. The method of claim 20, wherein said second oval-shaped opening and said hook are set apart at a distance dimensioned to allow passage and capture of the suture by the hook.

22. The method of claim 13, wherein said movable cutting blade comprises a front end having an angled blade surface and wherein said angled blade surface is configured to slide up and down the inclined stationary blade.

23. The method of claim 13, wherein the handle assembly further comprises a lever mechanism configured to secure the movable handle at a predetermined distance relative to the stationary handle, and wherein the lever mechanism comprises a lever terminating at one end at a lever button and having an opposite end pivotally connected to the stationary handle via a pivot pin and a spring mechanism.

24. The method of claim 23, wherein the lever further comprises a notch configured to engage a matching opposite notch in the back side of the movable handle and thereby to prevent movement of the movable handle.
